(19) United States
(12) Patent Application Publication
(10) Pub. No.: US 2013/0041428 A1
(43) Pub. Date: Feb. 14, 2013

(54) Upgradeable Cochlear Implant

(71) Applicant: Roger Leigh, Macquarie University (AU)

(72) Inventor: Roger Leigh, Macquarie University (AU)

(73) Assignee: COCHLEAR LIMITED, Macquarie University (AU)

(21) Appl. No.: 13/208,651

(22) Filed: Aug. 12, 2011

(51) Int. Cl. A61F 11/04; A61N 1/36

(52) U.S. Cl. 607/57

(57) ABSTRACT

The present application discloses an upgradeable cochlear implant. In one example, the implant may include a stimulator configured to generate a stimulation current comprising at least one stimulus, a first lead configured to conduct the stimulation current from the stimulator to at least one electrode, and a second lead configured to allow connection of a separate module to the stimulator. The second lead may include a lead conductor, a reference electrode configured to collect the stimulation current, an electrode conductor configured to conduct the stimulation current between the reference electrode and the stimulator, and an insulating material surrounding at least the lead conductor and the electrode conductor. The reference electrode may be formed at an exterior surface of the second lead, or may be formed on a portion of a connector for connecting to the separate module. The implant may additionally include a protective element.

### BACKGROUND

Cochlear implants may provide a person having sensorineural hearing loss with the ability to perceive sound by stimulating the person's auditory nerve via an array (or other configuration) of electrodes implanted in the person's cochlea. Typically, the cochlear implant functions to detect sound waves, convert the sound waves into a series of electrical stimulation signals, and deliver the stimulation signals to the cochlear implant recipient's auditory nerve via the array of electrodes. Stimulating the auditory nerve in this manner may enable the cochlear implant recipient's brain to perceive a hearing sensation that is similar to the natural hearing sensation delivered to a properly functioning auditory nerve.

Cochlear implants typically consist of two key components, namely an external component and an internal component. The external and internal components operate together to deliver the hearing sensation to the cochlear implant recipient.

The external component typically includes a microphone that detects sounds, such as speech and other environmental sounds, a speech processor that converts sounds detected by the microphone into a coded signal, a power source such as a battery, and an external transmitter unit. The internal component typically includes an internal receiver unit as well as a stimulator.

In operation, the external transmitter unit and the internal receiver unit may be positioned relative to one another so as to be inductively coupled. In this manner, data and power may be communicated transcutaneously from the external component to the internal component. This communication serves two essential purposes. First, the communication serves to transcutaneously transmit the coded sound signal output by the sound processor to the internal component and, second, the communication serves to provide power from the power source to the internal component. Conventionally, this link has been in the form of a radio frequency (RF) link, but other such links could also be implemented.

Once the coded signal is received by the internal component, the stimulator outputs a stimulation signal based on the coded signal to an array of electrodes implanted in the cochlea, and the array of electrodes applies the electrical stimulation to the auditory nerve of the cochlear implant recipient. The application of the electrical stimulation to the auditory nerve produces a hearing sensation that at least partially corresponds to the original detected sound.

The external component of the cochlear implant is typically carried on the body of the cochlear implant recipient, such as in a small unit worn behind the ear. While these so-called behind-the-ear (BTE) units are an improvement over previous cochlear implants, most cochlear implants still require an external transmitter unit to be positioned on the side of the cochlear implant recipient's head to allow for the transmission of the coded signal from the speech processor, and power from the power source, to the internal component.

The external component of the cochlear implant is a source of inconvenience and discomfort for many cochlear implant recipients. For example, cochlear implant recipients cannot wear the devices while showering or engaging in water-related activities. Most cochlear implant recipients also do not use the devices while sleeping due to discomfort caused by the presence of the BTE unit or the external transmitter unit and the likelihood that the alignment between the external transmitter unit and the internal receiver unit will be lost due to movements during sleep. For these and other reasons, there exists a need for a cochlear implant that allows for improved freedom, simplicity, and reliability.

One attractive option for meeting this need is a fully-implantable cochlear implant that allows the microphone, the power source, and the speech processor of the external component to be implanted in the cochlear implant recipient along with the internal component. In typical fully-implantable cochlear implants, one or more physical links are used to connect the stimulator of the internal component to the power source, the speech processor, and perhaps the microphone.

Generally, however, fully-implantable cochlear implants complicate the surgical procedure for implanting the cochlear implant by increasing the number of components that need to be implanted. In particular, the physical link(s) used to connect the stimulator to the power source, the speech processor, and the microphone typically require the addition of one or more leads on the stimulator. The one or more additional leads require a surgeon to drill additional recesses in which to house the new leads, thus increasing the complexity and length of the surgical procedure. Additionally, the increased length of the surgical procedure may increase the patient's risk of infection, as well as increase the costs of the surgery.

Thus, while fully-implantable cochlear implants are an improvement over typical cochlear implants in terms of a user's freedom and comfort, the added surgical complications may prevent fully-implantable cochlear implants from becoming a viable alternative to typical cochlear implants having one or more external components.

### SUMMARY

In an embodiment of the present application, the implantable device includes a stimulator, a first lead, and a second lead. The stimulator is configured to generate a stimulation current that includes at least one stimulus, and the first lead is configured to conduct the stimulation current from the stimulator to at least one electrode. The second lead is configured to allow connection of a separate module to the stimulator. The second lead includes a lead conductor, a reference electrode configured to collect the stimulation current at the electrode(s), an electrode conductor configured to conduct the stimulation current between the reference electrode and the stimulator, and an insulating material surrounding the lead conductor and the electrode conductor.

In one embodiment, the reference electrode is located at an exterior surface of the second lead. In this embodiment, the reference electrode may be patterned on some or all of the exterior surface of the second lead, or the reference electrode may be partially or entirely molded into the insulating material.

In a further embodiment, the second lead also includes a protective element that at least partially surrounds the insulating material. In this embodiment, the protective element may include a conductive material, and the reference electrode and the electrode conductor may be electrically coupled via the protective element. In this embodiment, the reference electrode may form a terminus for the protective element, such as a metallic ring or a clamp.

In still another embodiment, an additional insulating material partially surrounds the reference electrode such that a portion of the reference electrode is exposed by the additional insulating material. In this embodiment, the second lead may also include a protective element that at least partially surrounds the lead conductor and the electrode conductor and is at least partially surrounded by each of the reference electrode and the insulating material.

In at least one embodiment, the reference electrode is a mesh braid.

In an embodiment, the second lead also includes a portion of a connector for connecting to the separate module. In this embodiment, the reference electrode is located at an exterior surface of the portion of the connector.

In an embodiment, the implantable device is configured to be implanted in a body. As such, the reference electrode can include a bio-compatible conductive material and/or a noble metal.

In an embodiment, the separate module connectable to the second lead is one or both of a power source and a microphone.

A lead for an implantable device is also disclosed. In an embodiment, the lead includes a reference electrode, a lead conductor, an electrode conductor, and an insulating material. The reference electrode is configured to collect a stimulation current from at least one stimulation site. The lead conductor is configured to allow connection of a module to a stimulator. The electrode conductor is configured to conduct the stimulation current between the reference electrode and the stimulator, and the insulating material surrounds at least the lead conductor and the electrode conductor.

In one embodiment, the reference electrode is located at an exterior surface of the insulating material. In this embodiment, the reference electrode may be patterned on some or all of the exterior surface of the insulating material, or the reference electrode may be partially or entirely molded into the insulating material. In a further embodiment, the lead also includes a protective element that at least partially surrounds the insulating material.

In an embodiment, the reference electrode partially surrounds the lead conductor and the electrode conductor, and the insulating material partially surrounds the reference electrode such that a portion of the reference electrode is exposed by the insulating material.

In another embodiment, the lead also includes a portion of a connector for connecting to the module. In this embodiment, the reference electrode may be located at an exterior surface of the portion of the connector.

In an embodiment, the module connectable to the lead conductor is one or both of a power source and a microphone.

A cochlear implant is also disclosed. In an embodiment, the cochlear implant includes a stimulator configured to generate a stimulation current comprising a plurality of stimuli, and a first lead configured to conduct the stimulation current from the stimulator to at least one electrode. The cochlear implant also includes a second lead configured to allow connection of an implantable module to the stimulator. The second lead includes a lead conductor, a reference electrode configured to collect the stimulation current from the at least one electrode, an electrode conductor configured to conduct the stimulation current between the reference electrode and the stimulator, and a bio-compatible insulating material surrounding at least the lead conductor and the electrode conductor.

In an embodiment, the implantable module is one or both of a power source and a microphone.

### BRIEF DESCRIPTION OF THE DRAWINGS

FIG. 1 shows an example of a typical cochlear implant.

FIGS. 2A-2B show example internal components of typical cochlear implants, including a typical non-fully-implantable cochlear implant (FIG. 2A) and a typical upgradeable cochlear implant (FIG. 2B),

FIGS. 3A-3B show an overview of an example internal component of an upgradeable cochlear implant (FIG. 3A) and a lead from the example upgradeable cochlear implant (FIG. 3B), in accordance with the embodiment,

FIG. 4 shows a cross-sectional view of an example lead including a reference electrode formed at an exterior surface of an insulating material, in accordance with an embodiment,

FIGS. 5A-C show a cross-sectional view of an example lead including a reference electrode molded into an insulating material (FIG. 5A) and example boundaries for use in the example lead (FIGS. 5B-C), in accordance with another embodiment,

FIG. 6 shows a cross-sectional view of an example lead including a reference electrode that is partially exposed by an insulating material, in accordance with another embodiment,

FIG. 7 shows a cross-sectional view of an example lead including a protective element formed at an exterior surface of an insulating material and a reference electrode formed at an exterior surface of the protective element, in accordance with another embodiment,

FIG. 8 shows a cross-sectional view of an example lead including a protective element formed at an exterior surface of an insulating material and a reference electrode molded into the protective element, in accordance with another embodiment,

FIG. 9 shows a cross-sectional view of an example lead including an insulating material, a protective element, and a reference electrode that forms a ring terminus for the protective element, in accordance with another embodiment,

FIG. 10 shows a cross-sectional view of an example lead including an insulating material, a protective element, and a reference electrode that forms a clamp terminus for the protective element, in accordance with another embodiment,

FIG. 11 shows an example internal component of an upgradeable cochlear implant including a connector for connecting to an implantable module, in accordance with another embodiment,

FIG. 12 shows a cross-sectional view of an example lead and a portion of an example connector for connecting to an implantable module, in accordance with another embodiment, and

FIGS. 13A-B show example separate modules, including a power source (FIG. 13A) and a microphone (FIG. 13B) for connecting to an example cochlear implant, in accordance with an embodiment.

### DETAILED DESCRIPTION

The following detailed description describes various features and functions of the disclosed systems and devices with reference to the accompanying figures. In the figures, similar symbols typically identify similar components, unless context dictates otherwise. The illustrative embodiments described herein are not meant to be limiting. Certain aspects of the disclosed systems and devices can be arranged and combined in a wide variety of different configurations, all of which are contemplated herein. While the following description focuses on cochlear implants, it is to be understood that the description could apply to any number of implantable devices,

1. Cochlear Implant Overview

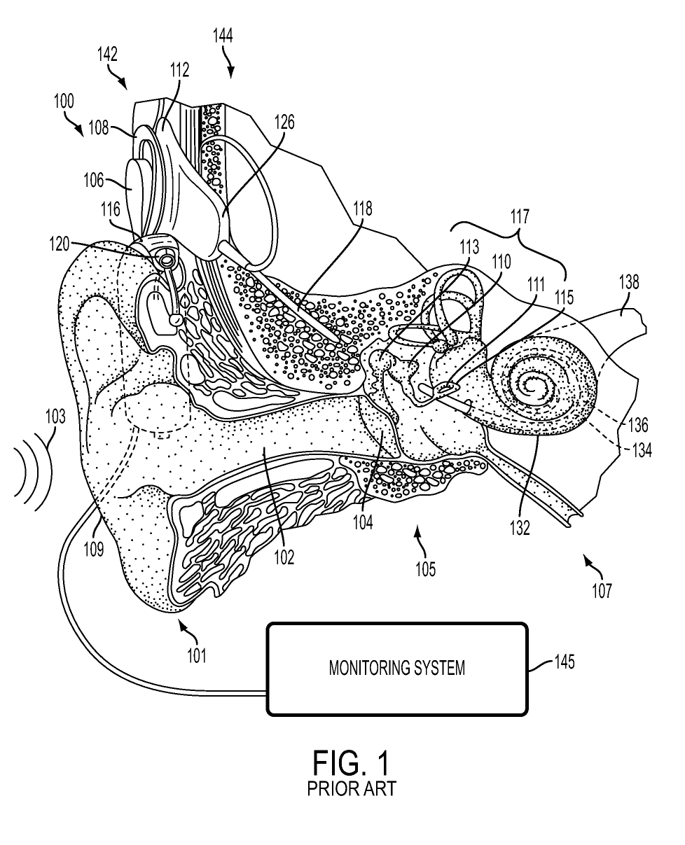

FIG. 1 shows an example of a cochlear implant. The relevant components of the recipient's outer ear 101, middle ear 105, and inner ear 107 are described, followed by a description of the cochlear implant 100.

For persons without certain types of hearing impairments, an acoustic pressure or sound wave 103 is collected by the auricle 109 and channeled into and through the ear canal 102. The tympanic membrane 104 is located at the distal end of the ear canal 102. The tympanic membrane 104 vibrates in response to the acoustic wave 103.

The vibration of the tympanic membrane 104 is coupled to the oval window or fenestra ovalis 115 through three bones of the middle ear 105, collectively referred to as the ossicles 117, and including the malleus 113, the incus 110, and the stapes 111. For persons without particular hearing impairments, the bones 113, 110, and 111 of the middle ear 105 serve to filter and amplify the acoustic wave 103, causing the oval window 115 to articulate and/or vibrate. The vibration of the oval window 115 causes waves of fluid motion within the cochlea 132. This fluid motion within the cochlea 132, in turn, activates tiny hair cells (not shown) that line the inside of the cochlea 132. Activation of the hair cells inside the cochlea 132 causes nerve impulses to be transferred through the spiral ganglion cells (not shown) and the auditory nerve 138 to the brain (not shown), where the nerve impulses may be perceived as sound. For persons with sensorineural hearing loss, a cochlear implant 100 may be used to create and apply electrical stimulation signals that may be similarly detected by a person's auditory nerve and perceived as sound.

The illustrated cochlear implant 100 includes an external component 142 that is directly or indirectly attached to the body of the recipient, and an internal component 144 that is implanted in the cochlear implant recipient.

The external component 142 includes a sound processor 116 and an external transmitter unit 106. The sound processor 116 includes a digital signal processor (DSP), a power source to power the cochlear implant 100, and a sound transducer 120. The sound transducer 120 is configured to detect sound and generate an audio signal, such as an analog audio signal, representative of the detected sound. In the example embodiment shown in FIG. 1, the sound transducer 120 is a microphone. In alternative embodiments, the sound transducer 120 can comprise, for example, more than one microphone, one or more telecoil induction pickup coils, or other devices now or later developed that detect sound and generate electrical signals representative of detected sound. In some embodiments, the sound transducer 120 may not be integrated into the sound processor 116, but rather could be a separate part of the external component 142.

The external transmitter unit 106 includes an external transmitter coil 108 along with associated circuitry to drive the coil. The external transmitter unit 106 also preferably includes a magnet (not shown) secured directly or indirectly to the external transmitter coil 108.

The sound processor 116 is configured to process the output of the microphone 120 in order to generate coded signals, which can be provided to the external transmitter unit 106 via a cable (not shown).

The internal component 144 includes an internal receiver unit 112, a stimulator 126, and a stimulator lead 118. The internal receiver unit 112 and the stimulator 126 are hermetically sealed within a bio-compatible housing.

The internal receiver unit 112 includes an internal receiver coil (not shown) along with the associated circuitry and is shown positioned in a recess of the temporal bone adjacent to the outer ear 101 of the recipient. The external transmitter coil 108 may be held in place and aligned with the implanted internal receiver coil via the above-referenced magnets. As set forth earlier, the external transmitter coil 108 is configured to transmit the coded signals from the sound processor 116 and power from the power source to the internal coil via a radio frequency (RF) link.

The stimulator lead 118 is designed to extend from the stimulator 126 to the cochlea 132 and to terminate in an array 134 of electrodes 136. Signals generated by the stimulator 126 are applied by the electrodes 136 to the cochlea 132, thereby stimulating the auditory nerve 138.

The illustrated cochlear implant 100 is further configured to interoperate with a cochlear implant monitoring system 145. The monitoring system 145 may include, for example, a computing device, such as a personal computer, a workstation, a handheld computing device, or the like. As shown, the cochlear implant 100 is connected to the monitoring system 145 via a wired connection or, in alternative embodiments, via a wireless connection (not shown).

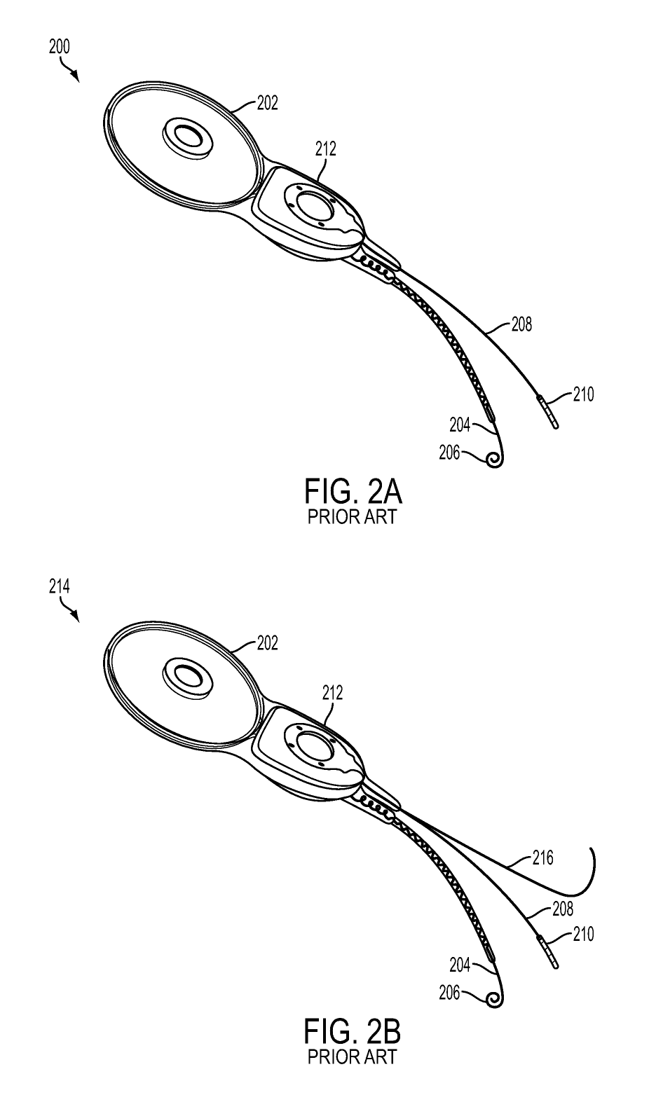

FIGS. 2A-2B show example internal components of typical cochlear implants. FIG. 2A depicts an internal component of a typical non-fully-implantable cochlear implant, such as that shown in FIG. 1. As shown, the internal component 200 includes an internal receiver unit 202 and a stimulator 212 to which two leads are connected.

The first lead 204 connects the stimulator 212 to one or more electrodes 206 (as described above). The electrode(s) 206 are designed to be implanted into the cochlea of a cochlear implant recipient. Signals generated by the stimulator 212 are transmitted to the electrodes 206 via the first lead 204 as, for example, a stimulation current. The electrode(s) 206 are in electrical communication with one or more stimulation sites in the cochlea, such that the stimulation current received at the electrode(s) 206 is applied to the stimulation site(s) to stimulate the auditory nerve, as described above.

The second lead 208 connects the stimulator 212 to a reference electrode 210 to provide a “return path” for the stimulation current from the stimulation site(s) back to the stimulator 212. The reference electrode 210 is similarly in electrical communication with the stimulation site(s) to collect the stimulation current applied by the electrode(s) 206. The stimulation current collected by the reference electrode 210 is conducted back to the stimulator 212 by the second lead 208.

In this manner, the stimulator 212, the first lead 204, the electrode(s) 206, the stimulation site(s), the reference electrode 210, and the second lead 208 form a circuit through which the stimulation current is conducted.

As noted above, one attractive option for improving the freedom of a cochlear implant recipient and the simplicity and reliability of cochlear implants is a fully-implantable cochlear implant, in which variations of both the internal and external components of the typical cochlear implant are implanted in the cochlear implant recipient.

In some cases, a cochlear implant recipient may receive a fully-implantable cochlear implant during a single surgery. That is, each of the stimulator, the microphone, the power source, the speech processor, and the one or more leads may be implanted during the single surgery. In other cases, the cochlear implant recipient may receive the components of the fully-implantable cochlear implant during two or more surgeries. For example, during a first surgery the cochlear implant recipient may receive an upgradeable cochlear implant. The upgradeable cochlear implant may function as and may be identical to a non-fully-implantable cochlear implant, with the exception that the stimulator of the upgradeable cochlear implant may be connected to an additional lead that terminates in a connector for connecting to a not-yet-implanted microphone, power source, and speech processor. Following the first surgery, the upgradeable cochlear implant may function as a non-fully-implantable cochlear implant, using an internal receiver unit to communicate with an external component, as described above. During a subsequent surgery, the implantable microphone, power source, and speech processor may be implanted in the cochlear implant recipient and connected to the additional lead. Following the subsequent surgery, the upgradeable cochlear implant may function as a fully-implantable cochlear implant, using the additional lead to communicate with the implanted microphone, power source, and speech processor.

The following description focuses on an upgradeable cochlear implant. It is to be understood, however, that the description could similarly apply to a fully-implantable cochlear implant (to be implanted during a single surgery, along with the implantable microphone, power source, and speech processor) as well. Such a fully-implantable cochlear implant may not include an internal receiver unit.

FIG. 2B depicts an internal component of a typical upgradeable cochlear implant. As shown, the internal component 214 of the typical upgradeable cochlear implant is similar to the internal component 200 of the non-fully-implantable cochlear implant, with the exception that the internal component 214 includes an additional lead 216 that terminates in a connector for connecting to, for example, a microphone, power source, and/or speech processor. Prior to implantation of the microphone, power source, and/or speech processor, the additional lead 216 may remain unconnected, and the upgradeable cochlear implant 214 may function as a non-fully-implantable cochlear implant, using the internal receiver unit 202 to communicate transcutaneously with an external component (not shown), as described above. Following implantation of the microphone, power source, and/or speech processor, however, the additional lead 216 may be used to connect the microphone, power source, and/or speech processor to the stimulator 212, and the upgradeable cochlear implant may function as a fully-implantable cochlear implant.

However, as noted above, the additional lead 216 included in the upgradeable cochlear implant typically requires a surgeon to drill an additional recess in the temporal bone of the cochlear implant recipient in which to house the additional lead 216, thus likely increasing the complexity, length, risk, and cost of the surgical procedure. In addition, the additional lead 216 may increase the complexity, length, and cost of the manufacturing process for manufacturing the typical upgradeable cochlear implant. Further, the additional lead 216 is an additional portion of the upgradeable cochlear implant that is susceptible to failure, thus reducing reliability of the typical upgradeable cochlear implant 212.

2. Upgradeable Cochlear Implant

a. Reference Electrode Formed on Additional Lead

For many potential cochlear implant recipients, the increased complexity, length, risk, and cost of the surgical procedure required for typical upgradeable cochlear implants may exceed the expected improvements in freedom, simplicity, and reliability offered by the typical upgradeable cochlear implant. Accordingly, it may, in many cases, be desirable to obtain the improvements offered by the upgradeable cochlear implant without increasing the complexity, length, risk, and/or cost of the surgical procedure. According to the present disclosure, an option for achieving this is to avoid an increase in the number of leads connected to the stimulator.

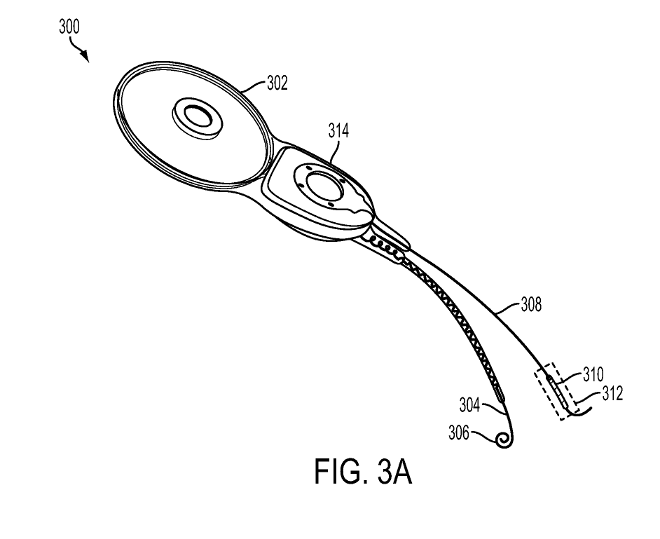

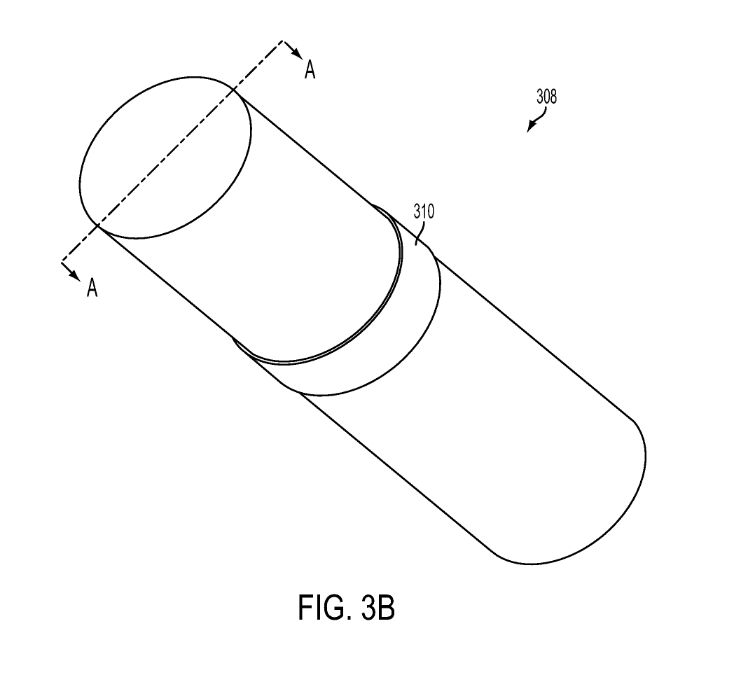

FIG. 3A shows an example internal component of an upgradeable cochlear implant, in accordance with an embodiment. As shown in FIG. 3A, the internal component 300 includes an internal receiver unit 302 and a stimulator 314 to which two leads are attached.

While the first lead 304 and the second lead 308 are shown to be approximately the same length, each of the first lead 304 and the second lead 308 can take various lengths, and the length of the first lead 304 can be the same as, greater than, or less than the length of the second lead 308. Additionally, the first lead 304 and the second lead 308 can each have various cross-sectional shapes of various dimensions, and the cross-section shapes and/or dimensions of the first lead 304 can be the same as or different than the second lead 308. In some embodiments, one or both of the first lead 304 and the second lead 308 can have, for example, a substantially cylindrical cross-section. In other embodiments, one or both of the first lead 304 and the second lead 308 can have, for example, a substantially rectangular cross-section. Other examples are possible as well.

The stimulator 314 is configured to generate a stimulation current. In one embodiment, the stimulation current is a pulsed current, where each pulse is a separate stimulus. In other embodiments, the stimulation current is an alternating current or a variable current. Other stimulation currents are possible as well.

The first lead 304 is configured to connect the stimulator 314 to one or more electrodes 306, as shown. As in typical cochlear implants, the first lead 304 is configured to conduct the stimulation current from the stimulator 314 to the one or more electrodes 306, and the electrode(s) 306 are configured to stimulate one or more corresponding stimulation sites.

The second lead 308 serves two purposes. The first purpose is to connect the stimulator 314 to a reference electrode 310, thereby providing a return path for the stimulation current, as described above. Additionally, however, the second lead 308 allows connection of a separate module to the internal component 300. In an embodiment, the separate module is a microphone and/or a power source. Other modules are possible as well.

By using a single lead to connect the stimulator 314 to both a reference electrode 310 and a separate module, the disclosed upgradeable cochlear implant includes a reduced number of leads compared to the typical upgradeable cochlear implant, thereby simplifying surgery and providing other benefits.

A detailed view of a portion 312 of the second lead 308, as indicated by the dotted line, is shown in FIG. 3B. As shown in FIG. 3B, the reference electrode 310 is formed around at least a portion of the outer perimeter of the second lead 308.

In some embodiments, the reference electrode 310 is configured to be located outside the cochlea, but still in electrical communication with the stimulation sites stimulated by the electrode(s) 306. As long as the reference electrode 310 is in electrical communication with the stimulation sites, the reference electrode 310 may provide a return path for the stimulation current applied to the stimulation sites. In some embodiments, the location of the reference electrode 310 on the second lead 308 is selected to achieve the desired location in the cochlear implant recipient.

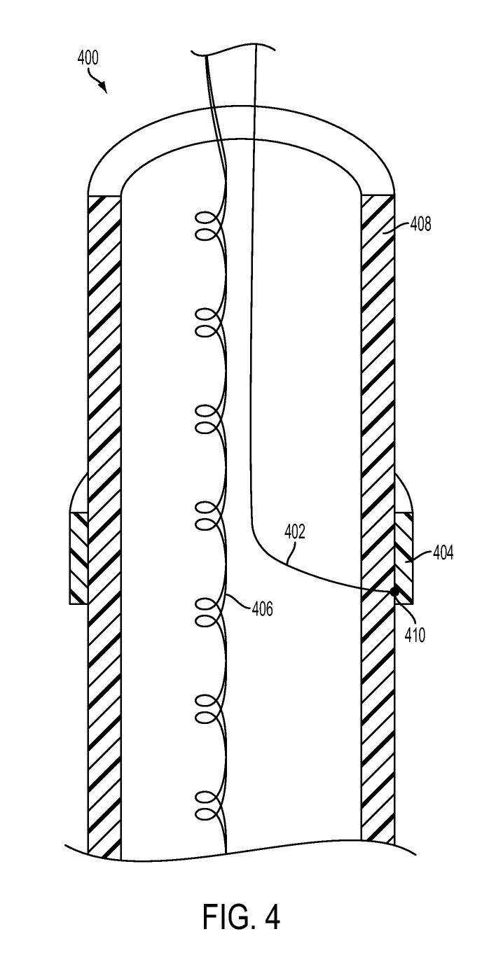

The reference electrode 310 may be formed on the second lead 308 in several configurations, some of which will be described below in connection with FIGS. 4-12. Each of FIGS. 4-12 is a cross-sectional perspective view of the second lead 308 cut along the line A-A in FIG. 3B.

FIG. 4 shows a cross-sectional view of an example lead including a reference electrode 404 formed at an exterior surface of an insulating material 408, in accordance with another embodiment. In some embodiments, the lead 400 serves to connect a stimulator (not shown), such as the stimulator 314 described above, to both the reference electrode 404 and a separate module (not shown). To this end, the lead 400 includes an electrode conductor 402 for conducting a stimulation current between the reference electrode 404 and the stimulator, as well as a lead conductor 406 for allowing connection of the separate module to the stimulator. The lead 400 also includes the insulating material 408 surrounding the electrode conductor 402 and the lead conductor 406. While the insulating material 408 is shown as a tube surrounding the electrode conductor 402 and the lead conductor 406, it is to be understood that this is merely illustrative and is not meant to be limiting, as the insulating material may take various forms besides that shown. In an embodiment, the insulating material 408 is molded around the electrode conductor 402 and the lead conductor 406 such that the insulating material 408 is in contact with electrode conductor 402 and the lead conductor 406. Other types of insulating material 408 are possible as well.

As shown, the top end of the lead 400 (where both the electrode conductor 402 and the lead conductor 406 are present) is connectable to the stimulator, while the bottom end of the lead 400 (where only the lead conductor 406 is present) is connectable to the separate module. However, other configurations of the stimulator, the separate module, and the conductors 402, 406 are possible as well.

As shown, the reference electrode 404 is located at an exterior surface of the lead 400. Additionally, the reference electrode 404 is electrically coupled to the electrode conductor 402 at a contact point 410. In one embodiment, the reference electrode 404 and the electrode conductor 402 are joined using one or more joining processes, such as resistance welding or laser welding. Other joining processes are possible as well.

In some embodiments, the electrode conductor 402 includes one or more electrically conductive materials, such as platinum, iridium, or gold. If the material used for the electrode conductor 402 is a non-bio-compatible material, such as silver, the lead conductor 406 may be encased in a bio-compatible material, such as stainless steel. Other examples are possible as well. The electrode conductor 402 may be straight, helixed, or a combination of straight and helixed. In some embodiments, the electrode conductor 402 may be partially or wholly surrounded by an insulating material.

In some embodiments, the reference electrode 404 includes one or more electrically conductive materials. Examples may include both noble metals (such as platinum) and non-noble metals (such as titanium). In some embodiments, some or all of the electrically conductive material(s) are bio-compatible. The reference electrode 404 may extend around the entire outer perimeter of the lead 400, or may extend around only a portion of the outer perimeter of the lead 400. The reference electrode 404 may take a variety of shapes. As an example, in embodiments where the lead 400 has a substantially cylindrical cross-section, the reference electrode 404 may be arc-shaped or circular. As another example, in embodiments where the lead 400 has a substantially rectangular cross-section, the reference electrode 404 may be linear and/or rectangular. As still another example, the reference electrode 404 may be designed to fit flush against an exterior surface of the lead 400. Other examples are possible as well.

Additionally, the reference electrode 404 may take on a variety of dimensions and thicknesses. In some embodiments, the dimensions and thicknesses of the reference electrode 404 are selected based on, for example, a desired current density of the stimulation current, the material(s) included in the reference electrode 404, and/or a desired location on the lead 400. In some embodiments, the exterior surface of the reference electrode 404 is processed to alter its effective surface area. As an example, in an embodiment the exterior surface of the reference electrode 404 is subjected to surface roughening. Other examples are possible as well.

Similarly, the reference electrode 404 may extend along the entire longitudinal length of the lead 400, or may extend along only a portion of the longitudinal length of the lead 400. Additionally, the reference electrode 404 may be positioned anywhere along the longitudinal length of the lead 400, and the position of the reference electrode 404 may vary.

In some embodiments, the reference electrode 404 is patterned onto the exterior surface of the lead 400 through, for example, a sputtering process. In other embodiments, the reference electrode 404 is pre-formed and attached to the exterior surface of the lead 400 using one or more joining processes, such as resistance welding or laser melding.

In some embodiment, the lead conductor 406 similarly includes one or more electrically conductive materials, such as platinum, iridium, or gold. If the material used for the lead conductor 406 is a non-bio-compatible material, such as silver, the lead conductor 406 may be encased in a bio-compatible material, such as stainless steel. Other examples are possible as well. The lead conductor 406 may be straight, helixed, or a combination of straight and helixed. The lead conductor 406 preferably comprises two or more conductors (i.e., wires), depending on the application of the lead 400, in order to provide a complete circuit for data and/or power transmission. In some embodiments, the lead conductor 406 may be partially or wholly surrounded by an insulating material.

In some embodiments, the insulating material 408 includes one or more electrically insulating materials, such as silicone rubber, polyether ether ketone (PEEK), ceramic, plastic, or other insulating materials. The insulating material 408 may take a variety of shapes and a variety of dimensions and thicknesses.

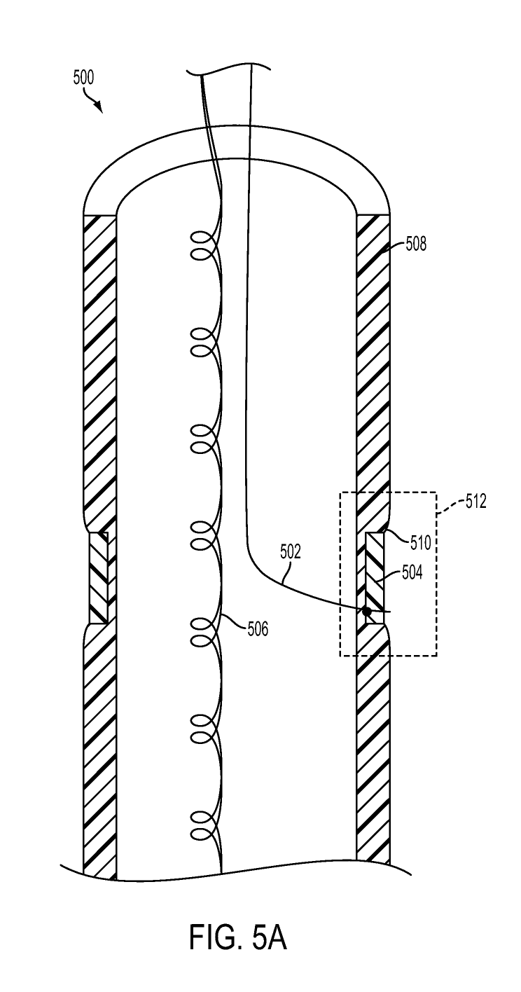

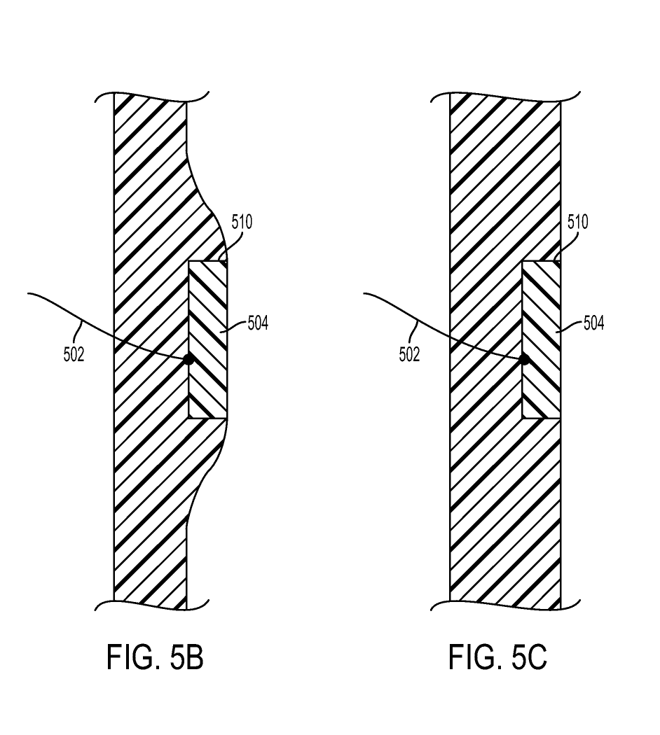

FIG. 5A shows a cross-sectional view of an example lead including a reference electrode molded into an insulating material, in accordance with another embodiment. As shown, the lead 500 is similar to the lead 400 of FIG. 4 in that the lead 500 includes an electrode conductor 502, a reference electrode 504, a lead conductor 506, and an insulating material 508 surrounding the electrode conductor 502 and the reference electrode 504. As shown, however, the reference electrode 504 is molded into (or embedded in) the insulating material 508. In this configuration, an exterior surface of the reference electrode 504 is exposed so that it may collect a stimulation current from one or more stimulation sites, as described above, while an opposing surface and at least a portion of the side surfaces are embedded in the insulating material 508.

In some embodiments, such as that shown in FIG. 5A, the insulating material 508 extends along the sides of the reference electrode 504 as far as the surface of the reference electrode 504. In other embodiments, the insulating material 508 extends only a portion of the way along the sides of the reference electrode 504. Additionally, in some embodiments, such as that shown in FIG. 5A, the insulating material 508 extends on both sides of the reference electrode 504. In other embodiments, the insulating material 508 only extends on one side of the reference electrode 504. In some embodiments, the reference electrode 504 does not protrude beyond an exterior surface of the lead 500, but rather is molded in the insulating material 508 so that the exterior longitudinal surface of the lead 500 is substantially smooth.

While a boundary 510 is shown to be an inward tapered boundary, it is to be understood that this boundary 510 is merely illustrative and is not meant to be limiting. Other possible boundaries 510 are shown in FIGS. 5B and 5C. In particular, FIGS. 5B-C depict a portion 512 of the lead 500 shown in FIG. 5A. As shown in FIG. 5B, the boundary 510 may, in some embodiments, be an outward tapered boundary. Further, as shown in FIG. 5C, the boundary may, in some embodiments, be a flat boundary. Other boundaries are possible as well. In general, the boundary 510 may be selected to be any kind of smooth boundary that allows for a flush and smooth outer surface of the lead 500, thus minimizing the risk of bacterial or biofilm growth on the lead 500.

In some embodiments, instead of being molded into the insulating material 508 itself, the reference electrode 504 is molded in a different material that at least partially surrounds the insulating material 508. This may be desirable in embodiments where the insulating material 508 does not have one or more material properties that are desirable for molding the reference electrode 504 in the insulating material 508.

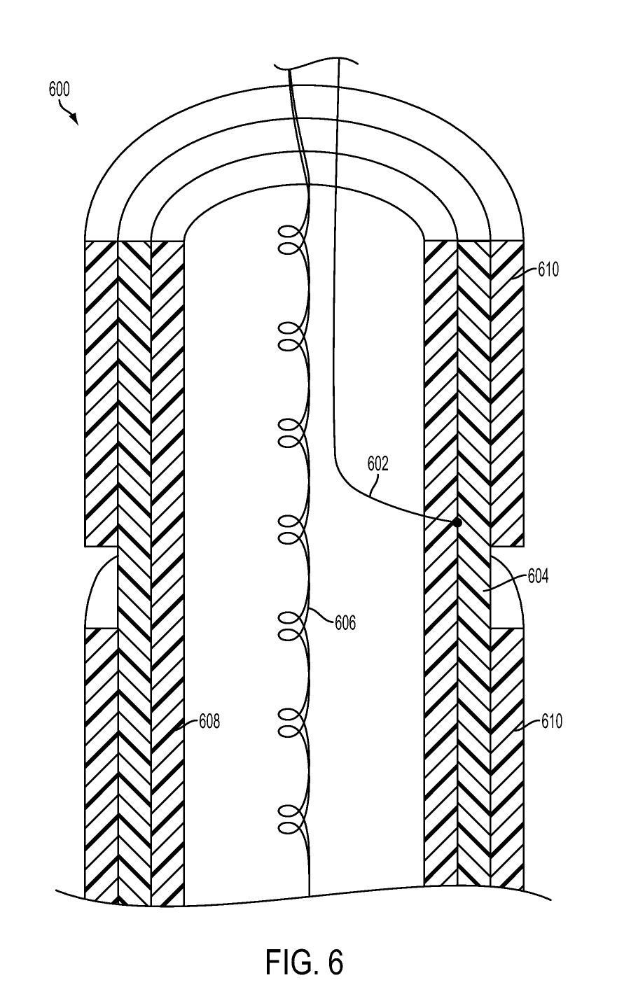

In the embodiments shown in FIGS. 4 and 5A-C, the insulating material is present between the electrode and lead conductors and the reference electrode. In some embodiments, however, the reference electrode surrounds the electrode conductor and the lead conductor, and the insulating material partially surrounds the reference electrode such that a portion of the reference electrode is exposed by the insulating material. FIG. 6 shows a cross-sectional view of an example lead including a reference electrode that is partially exposed by an insulating material, in accordance with another embodiment.

In FIG. 6, the lead 600 includes an electrode conductor 602 in electrical contact with a reference electrode 604, a lead conductor 606, and an insulating material 608. The lead 600 additionally includes an additional insulating material 610 that partially surrounds the reference electrode 604 such that a portion of the reference electrode 604 is exposed by the insulating material 610. The exposed portion of the reference electrode 604 is positioned to be in electrical communication with one or more corresponding stimulation sites, so as to collect the stimulation current, as described above.

As shown, while the reference electrode 604 is exposed along only a portion of the length of the lead 600, in some embodiments the reference electrode 604 extends along a much larger longitudinal portion of the lead 600, and, in some embodiments, extends along the entire longitudinal length of the lead 600. This configuration offers a benefit in that the reference electrode 604 may serve as a sort of protective element providing protection to the electrode conductor 602 and the lead conductor 606. Such protection is valuable during, for example, implantation of an upgradeable cochlear implant during which time the electrode conductor 602 and the lead conductor 606 may be at risk of harm by one or more sharp or pointed surgical tools, or by other means.

In some embodiments, in order to achieve further protection, it may be desirable to extend the reference electrode 604 around the full perimeter of the lead 600, as well as along a larger portion of (or the entire) longitudinal length of the lead 600. However, the reference electrode 604 may offer protection even if the reference electrode 604 does not extend fully around or fully along the longitudinal length of the lead 600.

In some embodiments, in order to achieve further protection, the reference electrode 604 is designed in a form that is more resistant to sharp or pointed surgical tools, such as a braided metal design. A braided metal design allows the insulating material 608 to flow through the reference electrode 604 (such as during manufacture), thereby “locking” it in place. Other examples are possible as well.

While the reference electrode itself may serve as a protective element, as described in connection with FIG. 6, in some embodiments the lead includes a separate protective element. The separate protective element protects the electrode and lead conductors instead of or in addition to the reference electrode at various positions along the length of the lead. In some embodiments, the separate protective element is selected to be equally or more resistant to sharp or pointed surgical tools, as compared with the reference electrode.

In some embodiments, the protective element partially or fully extends around the perimeter of the lead, and extends along some or all of the length of the lead. As with the reference electrode, in order to achieve further protection, it may be desirable to extend the protective element all the way around the perimeter of the lead, as well as along a larger portion of (or the entire) length of the lead.

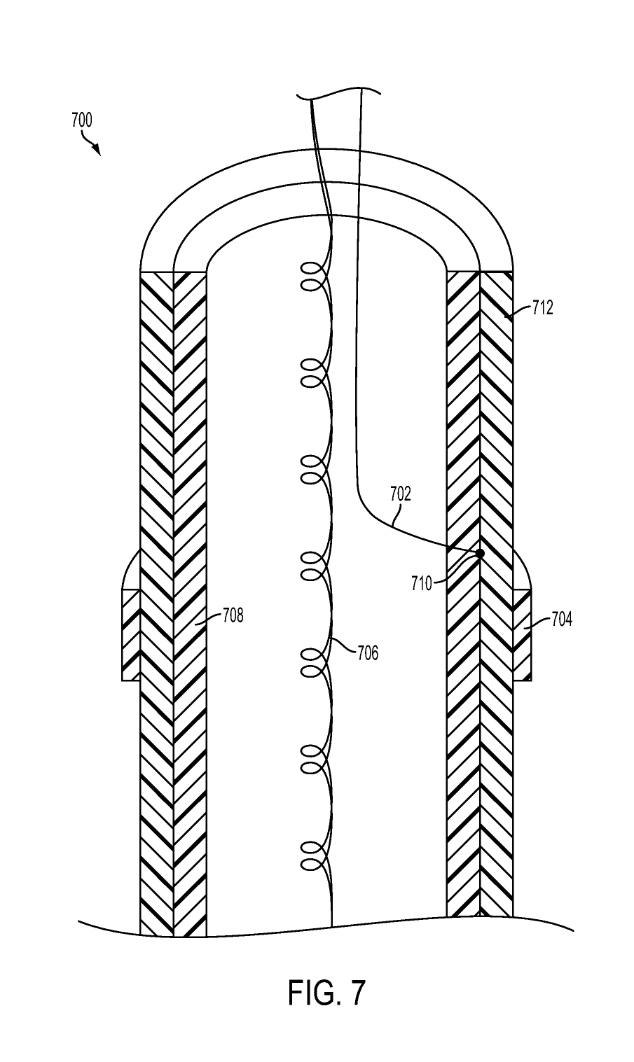

FIG. 7 shows a cross-sectional view of an example lead including a protective element formed at an exterior surface of an insulating material and a reference electrode formed at an exterior surface of the protective element, in accordance with another embodiment. As shown, the lead 700 includes an electrode conductor 702, a reference electrode 704, a lead conductor 706, an insulating material 708, and a protective element 712. The reference electrode 704 is shown positioned at an exterior surface of the protective element 712, which may allow the reference electrode 704 to collect a stimulation current from one or more corresponding stimulation sites, as described above.

In some embodiments, the protective element 712 includes one or more of a stent, a helixed spring, a tube surrounding the insulating material 708, or another protective element. In some embodiments, such as the one shown, the protective element 712 includes one or more electrically conductive materials.

In embodiments where the protective element 712 includes an electrically conductive material, the electrode conductor 702 and the reference electrode 704 may be electrically coupled via the protective element 712. In the embodiment shown, a contact point 710 between the electrode conductor 702 and the reference electrode 704 is located on an interior edge of the protective element 712. Because the protective element 712 includes a conductive material, current (such as the stimulation current) may flow from the reference electrode 704 through the protective element 712 to the electrode conductor 702 and back to a stimulator (not shown). Alternately, in embodiments where the protective element 712 includes an electrically conductive material, the protective element 712 may serve as the reference electrode 704 itself, similar to the embodiment described above in connection with FIG. 6.

In embodiments where the protective element 712 does not include an electrically conductive material, the contact point 710 may be located elsewhere, such as on an exterior edge of the protective element 712, or on an interior edge of the insulating material 708. In either case, one or both of the electrode conductor 702 and the reference electrode 704 may extend into and/or through the protective element 712 and/or the insulating material 708 to meet at the contact point 710.

The reference electrode 704 may be patterned onto the exterior surface of the protective element 712, or may be pre-formed and attached to the exterior surface of the protective element 712 using one or more joining processes, as described above.

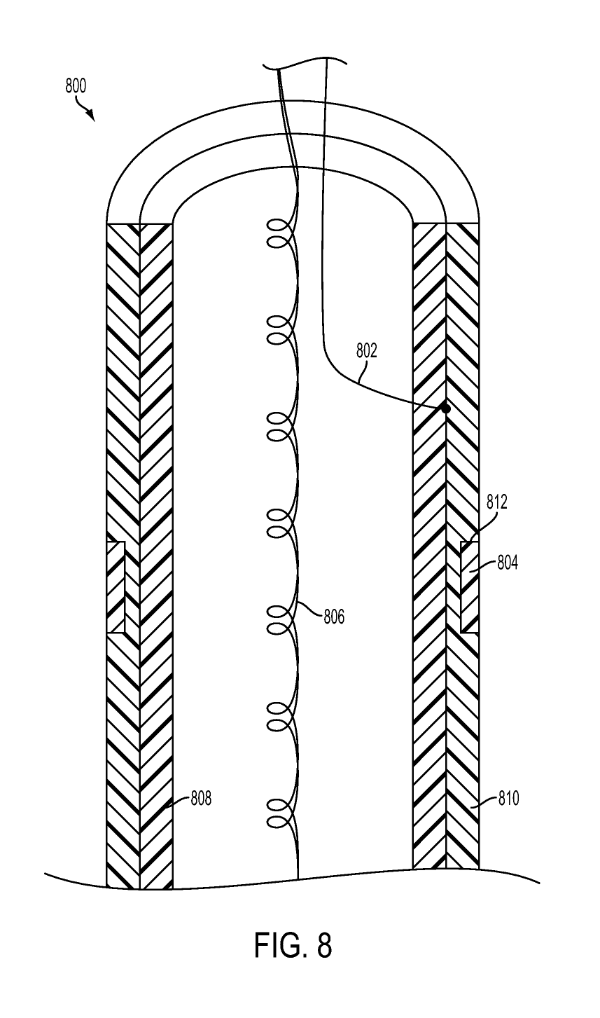

FIG. 8 shows a cross-sectional view of an example lead including a protective element formed at an exterior surface of an insulating material and a reference electrode molded into the protective element, in accordance with another embodiment. The lead 800 is similar to the lead 700 of FIG. 7 in that the lead 800 includes an electrode conductor 802, a reference electrode 804, a lead conductor 806, an insulating material 808, and a protective element 810.

As shown in FIG. 8, however, the reference electrode 804 is molded into the protective element 810 such that only an exterior surface of the reference electrode 804 is located at an exterior surface of the lead 800. A transition 812 between the reference electrode 804 and the protective element 814 is shown to be smooth to minimize the risk of bacterial and biofilm growth on the lead 800, though other transitions are possible as well.

In some embodiments, such as that shown in FIG. 8, the protective element 810 extends along the sides of the reference electrode 804 as far as the external surface of the reference electrode 804. In other embodiments, the protective element 810 extends only a portion of the way along the sides of the reference electrode 804. Additionally, in some embodiments, such as that shown, the protective element 810 extends on both sides of the reference electrode 804. In other embodiments, the protective element 810 only extends on one side of the reference electrode 804.

In some embodiments, instead of being molded into the protective element 810 itself, the reference electrode 804 is molded in a different material that at least partially surrounds the protective element 810. This may be desirable in embodiments where the protective element 810 does not have one or more material properties that are desirable for molding the reference electrode 804 in the protective element 810.

While a protective element may serve to protect the electrode conductor and the lead conductor in a lead, the protective element may also be susceptible to problems such as fraying that may result in exposure of sharp edges. In order to prevent such fraying, in some embodiments it may be desirable to provide a terminus for the protective element. In some embodiments, the terminus surrounds the end of the protective element such that any sharp edges on the protective element touch only the terminus and not the electrode and lead conductors.

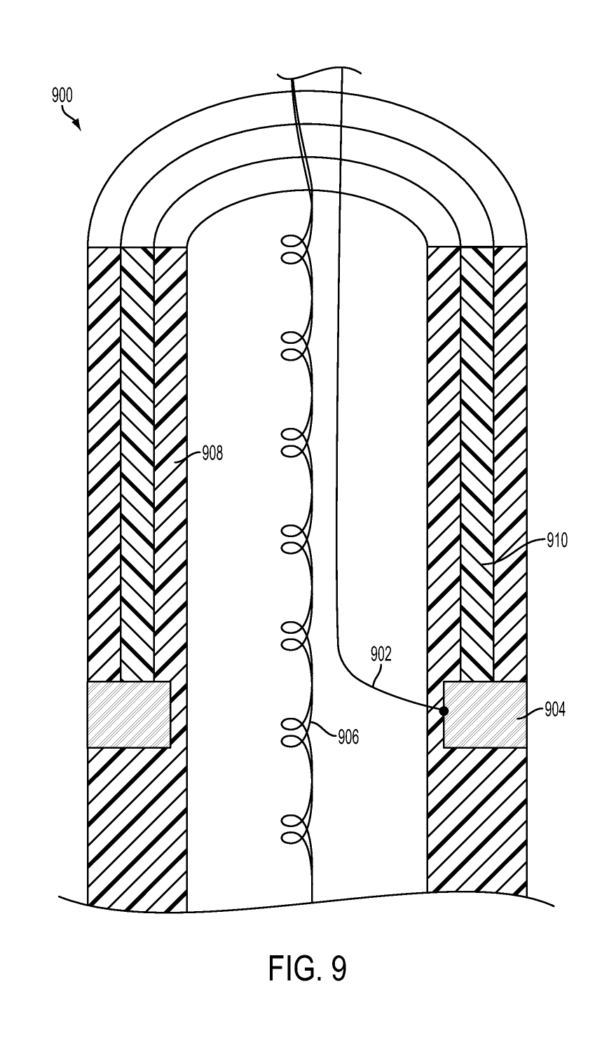

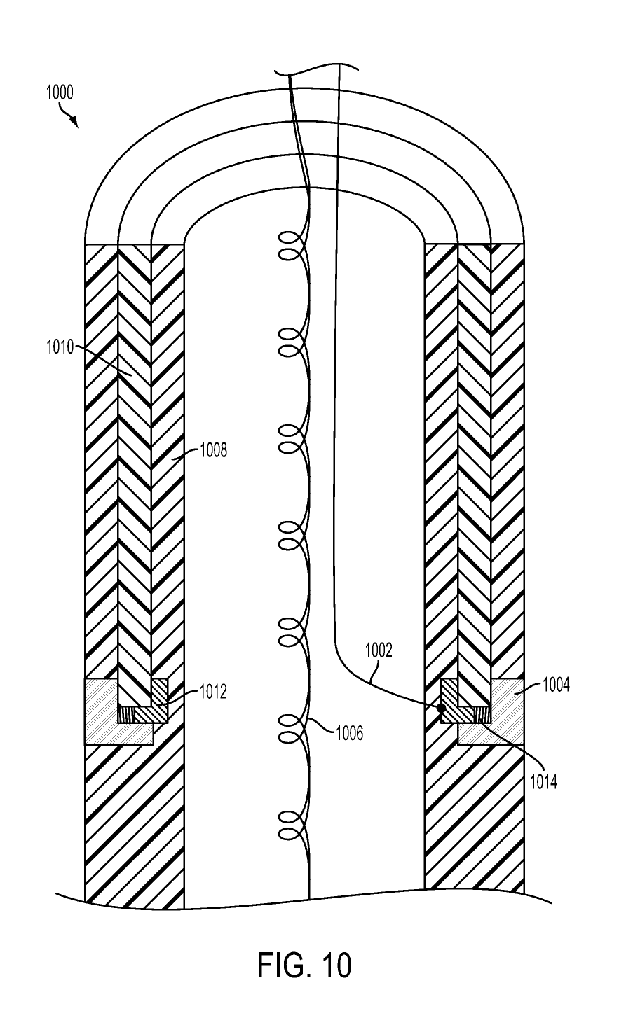

Rather than introducing an additional element into the lead, which may complicate the manufacturing, use, and reliability of the lead, in some embodiments the reference electrode itself is formed as a terminus for the protective element. Various types of termini are possible. Two examples are shown in FIGS. 9-10, though other examples are possible as well.

FIG. 9 shows a cross-sectional view of an example lead including an insulating material, a protective element, and a reference electrode that forms a ring terminus for the protective element, in accordance with another embodiment. The lead 900 includes an electrode conductor 902, a reference electrode 904, a lead conductor 906, an insulating material 908, and a protective element 910. In the example shown, the insulating material 908 also surrounds a portion of the protective element 910.

As shown, the reference electrode 904 surrounds the end of the protective element 912, thereby reducing the risk of exposed sharp edges at the end of the protective element 910. Additionally, an exterior edge of the reference electrode 904 is exposed such that the reference electrode 904 may collect a stimulation current from one or more corresponding stimulation sites, as described above.

Another example of a terminus is a clamp terminus. FIG. 10 shows a cross-sectional view of an example lead including an insulating material, a protective element, and a reference electrode that forms a clamp terminus for the protective element, in accordance with another embodiment. The lead 1000 includes an electrode conductor 1002, a reference electrode 1004, a lead conductor 1006, an insulating material 1008, a protective element 1010, and an electrically conductive ring 1012. The electrode conductor 1002 and the reference electrode 1004 are shown to be electrically coupled via the electrically conductive ring 1012. Alternately or additionally, in embodiments where the protective element 1010 is electrically conductive, the electrode conductor 1002 and the reference electrode 1004 may be electrically coupled via the protective element 1010.

As shown, the protective element 1010 is clamped between the reference electrode 1004 and the electrically conductive ring 1012. In some embodiments, the distance between the reference electrode 1004 and the electrically conductive ring 1012 is controlled using, for example, a thread 1014 or other mechanism.

In some embodiments, the electrically conductive ring 1012 includes one or more electrically conductive materials, such as titanium, a titanium alloy, or stainless steel. The electrically conductive ring 1012 may be a full ring, such that it fully extends around the perimeter of the lead 1000, or may be an arc or partial ring, such that it extends around a portion of the perimeter of the lead 1000 but is not complete. Similarly, the electrically conductive ring 1012 may extend along some or all of the length of the lead 1000. Other types of termini besides those shown in FIGS. 9-10 are possible as well.

b. Reference Electrode Formed on Connector

As noted above, the additional lead may be configured to allow connection of one or more separate modules to the stimulator in a cochlear implant. To this end, in some embodiments, the additional lead includes a portion of a connector for connecting to one or more separate modules. The one or more separate modules may similarly include a portion of the connector that is connectable to the portion of the connector included on the lead.

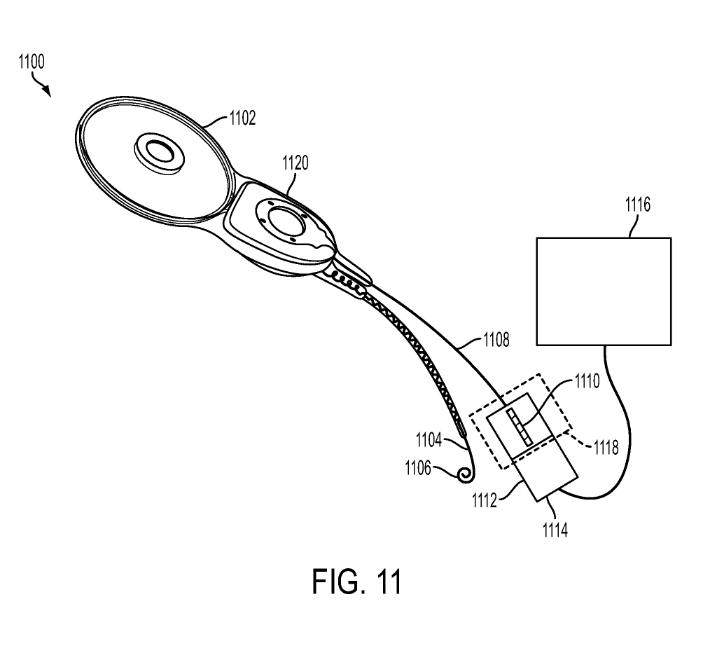

FIG. 11 shows an example internal component of an upgradeable cochlear implant including a connector for connecting to an implantable module, in accordance with another embodiment. The internal component 1100 is similar to the internal component 300 shown in FIG. 3, in that it includes an internal receiver unit 1102 and a stimulator 1120 to which two leads are attached. Additionally, however, the internal component 1100 is shown to be connected to a separate, implantable module 1116. The upgradeable cochlear implant may include other components in addition to or instead of those shown.

While the first lead 1104 and the second lead 1108 are shown to be approximately the same length, the length of each of the first lead 1104 and the second lead 1108 may vary, and the length of the first lead 1104 may be the same as, greater than, or less than the length of the second lead 1108. Additionally, the first lead 1104 and the second lead 1108 may each have various cross-sectional shapes of various dimensions, and the cross-sectional shape and/or dimensions of the first lead 1104 may be the same as or different than those of the second lead 1108. In some embodiments, one or both of the first lead 1104 and the second lead 1108 may have, for example, a substantially circular cross-section. In other embodiments, one or both of the first lead 1104 and the second lead 1108 may have, for example, a substantially rectangular cross-section. Other examples are possible as well.

The first lead 1104 connects the stimulator 1120 to one or more electrodes 1106. As in typical cochlear implants, the first lead 1104 is configured to conduct the stimulation current from the stimulator 1120 to the one or more electrodes 1106.

The second lead 1108 serves two purposes. The first purpose is to connect the stimulator 1120 to a reference electrode 1110, thereby providing a return path for the stimulation current, as described above. Additionally, however, the second lead 1108 allows connection of the implantable module 1116 to the stimulator 1120. In some embodiments, this connection is made through a first portion of a connector 1118 and a second portion of a connector 1114. In some embodiments, the implantable module 1116 is a microphone and/or a power source. Other modules are possible as well.

By using a single lead to connect the stimulator 1120 to both the reference electrode 1110 and the implantable module 1116, the upgradeable cochlear implant improves upon the typical fully-implantable cochlear implant without increasing the number of leads connected to the stimulator 1120.

In contrast to the upgradeable cochlear implant shown in FIG. 3, however, the dual-purpose second lead 1108 is made possible through the placement of the reference electrode 1110 on the first portion of the connector 1118, rather than at an exterior surface of the second lead 1108 itself. The placement of the reference electrode 1110 on the first portion of the connector 1118 may take many configurations. In some embodiments, the reference electrode 1110 is patterned onto, attached to, or molded into an exterior surface of the first portion of the connector 1118, or the case of the first portion of the connector 1118 may be formed of an electrically conductive material, such as titanium, that may be partially or fully exposed to form the reference electrode. Other examples are possible as well.

In some embodiments, the reference electrode 1110 is configured to be located outside the cochlea, but still in electrical communication with the stimulation sites stimulated by the electrode(s) 1106. This electrical communication is typically achieved via tissue, bone, and/or body fluids within an implant recipient. As long as the reference electrode 1110 is in electrical communication with the stimulation sites, the reference electrode 1110 provides a return path for the stimulation current applied to the stimulation sites. In some embodiments, the location of the reference electrode 1110 on the second lead 1108 is selected to achieve the desired location in the cochlear implant recipient.

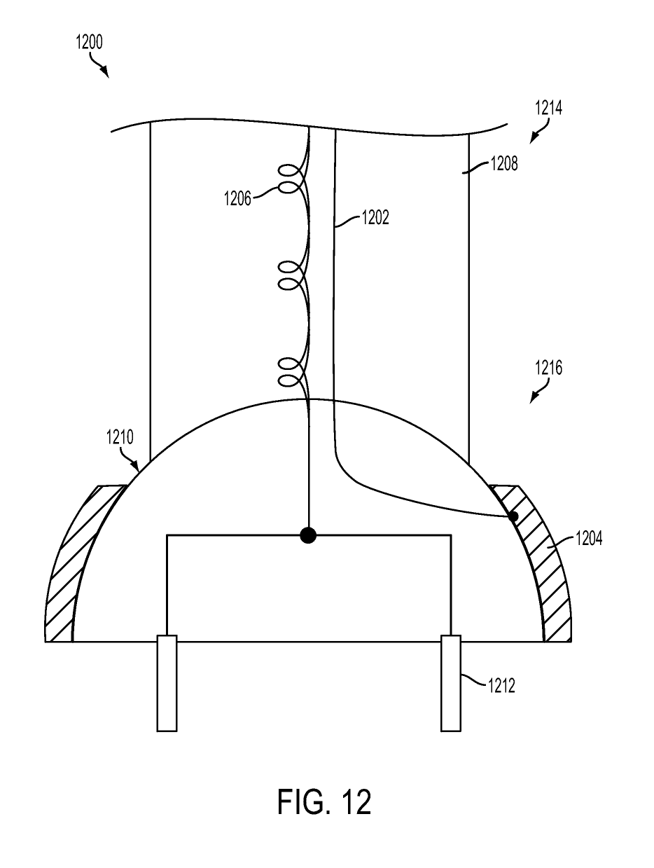

FIG. 12 is a detailed view of a portion 1118 of the second lead 1108, as indicated by the dotted line in FIG. 11. FIG. 12 shows a cross-sectional view of an example lead and a portion of an example connector for connecting to an implantable module, in accordance with another embodiment. The lead 1200 includes an electrode conductor 1202 that is electrically coupled to a reference electrode 1204, a lead conductor 1206, and an insulating material 1208.

A top end 1214 of the lead 1200 is connectable to the stimulator, and a bottom end 1216 of the lead 1200 is connectable to the implantable module. To this end, the lead 1200 is shown to terminate in a portion of a connector 1210. The portion of the connector 1210 allows connection of the stimulator to one or more implantable modules.

The reference electrode 1204 may be positioned at an exterior surface of the portion of the connector 1210, which may allow the reference electrode 1204 to collect a stimulation current at one or more corresponding stimulation sites, as described above. To this end, the reference electrode 1204 may be patterned onto the exterior surface of the portion of the connector 1210 using one or more patterning processes, as described above, or may be pre-formed and attached to the exterior surface of the portion of the connector 1210 using one or more joining processes, such as resistance welding or laser welding.

In some embodiments, the reference electrode 1204 extends around the entire perimeter of the portion of the connector 1210, or extends around only a portion of the perimeter of the portion of the connector 1210. Similarly, in some embodiments, the reference electrode 1204 extends along the entire height and perimeter of the portion of the connector 1210, or extends along only a portion of the height and perimeter of the portion of the connector 1210. Additionally, in some embodiments, the reference electrode 1204 is positioned anywhere along the height of the portion of the connection 1210, and the position of the reference electrode 1204 may vary.

The portion of the connector 1210 may take a variety of dimensions and cross-sectional shapes. In some embodiments, the portion of the connector 1210 has a substantially circular cross-section. In other embodiments, the portion of the connector 1210 has a substantially rectangular cross-section. Other examples are possible as well. In some embodiments, the portion of the connector 1210 is formed of one or more electrically conductive materials, such as titanium, and/or of one or more non-conductive materials, such as polyether ether ketone (PEEK).

Similarly, the reference electrode 1204 may take a variety of shapes of a variety of dimensions and thicknesses. As an example, in embodiments where the portion of the connector 1210 has a substantially circular cross-section, the reference electrode 1204 may be arc-shaped or circular. As another example, in embodiments where the portion of the connector 1210 has a substantially rectangular cross-section, the reference electrode 1204 may be rectangular. As still another example, in some embodiments the reference electrode 1204 is designed to fit flush against the exterior surface of the portion of the connector 1210. In still other embodiments, when the first portion of the connector 1210 is formed of an electrically conductive material, such as titanium, the electrically conductive material may be partially or fully exposed to form the reference electrode 1204. Other examples are possible as well.

As shown, the lead conductor 1206 terminates inside the portion of the connector 1210, and the portion of the connector 1210 includes two prongs 1212 for connecting to another portion of the connector (e.g., a portion of the connector associated with an implantable module). In some embodiments, the prongs 1212 are used to, for example, transfer power and/or data between the stimulator and the implantable module. It is to be understood that the portion of the connector 1210 shown is merely one embodiment, and that many other types of connectors could be used as well. Examples of connectors include plug and socket type connectors, blade connectors, ring and spade connectors, and terminal blocks. Other connectors are possible as well. It is to be further understood that the illustration of the portion of the connector 1210 and the circuitry within the portion of the connector 1210 are merely illustrative, and that the circuitry may vary depending on the type (and gender) of the portion of the connector 1210.

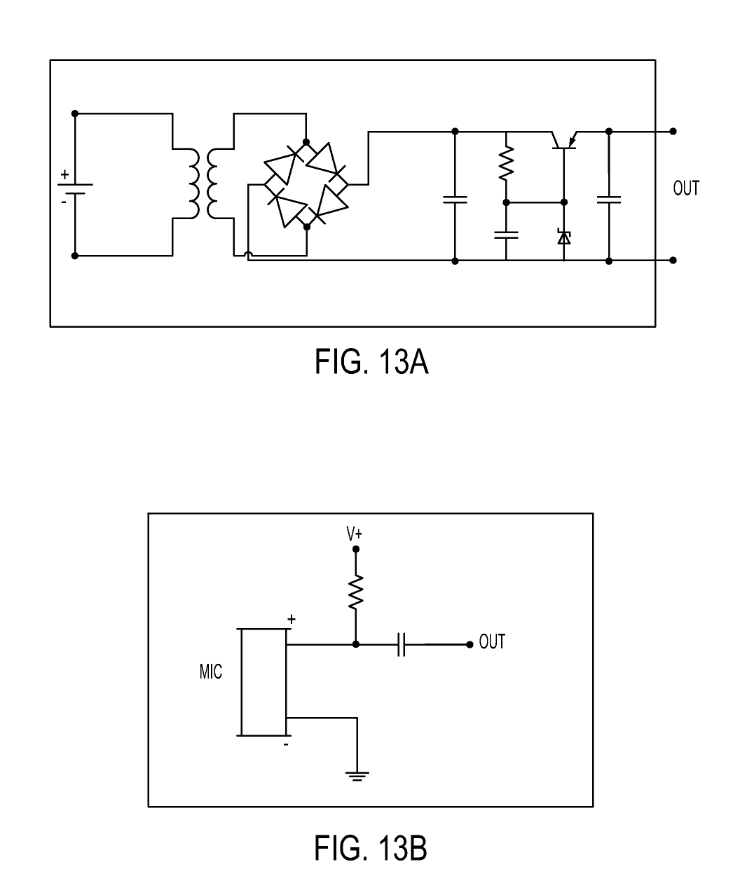

FIGS. 13A-B show example separate modules, including a power source (FIG. 13A) and a microphone (FIG. 13B) for connecting to an example cochlear implant, in accordance with an embodiment. FIG. 13A shows an example circuit diagram of a basic power source, while FIG. 13B shows an example circuit diagram of a basic microphone. The circuits shown are merely illustrative and are not meant to be limiting, and each of the circuits may comprise additional elements in addition to or instead of those shown. In some embodiments, the separate modules may be implantable modules. In some embodiments, the separate module includes both a power source and a microphone. In other embodiments, the cochlear implant may include a connector that is connectable to both a power source and a microphone. Other embodiments are possible as well.

It is to be understood that the configurations shown in the figures are merely illustrative and are not intended to be limiting. In particular, the sizes, shapes, and positions of the elements shown in the figures are merely illustrative, and other sizes, shapes and positions are possible as well. Further, the various features of the configurations shown in the figures may be added, removed, combined, or otherwise modified to result in many more configurations that are similarly contemplated.

While the foregoing description focuses on cochlear implants, several features of the systems and devices disclosed herein could be used in any number of other implantable stimulation systems including but not limited to auditory brainstem implant systems, deep brain stimulation systems, and mid-brain stimulation systems. Other types of implantable systems are possible as well.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. An implantable device, comprising:
a stimulator configured to generate a stimulation current comprising at least one stimulus;
a first lead configured to conduct the stimulation current from the stimulator to at least one electrode;
a second lead configured to allow connection of a separate module to the stimulator, wherein the second lead comprises (i) a lead conductor, (ii) a reference electrode configured to collect the stimulation current at the at least one electrode, (iii) an electrode conductor configured to conduct the stimulation current between the reference electrode and the stimulator, and (iv) an insulating material surrounding at least the lead conductor and the electrode conductor.

2. The implantable device of claim 1, wherein the reference electrode is located at an exterior surface of the second lead.

3. The implantable device of claim 2, wherein the reference electrode is patterned over at least a portion of the exterior surface of the second lead.

4. The implantable device of claim 2, wherein the reference electrode is at least partially molded into the insulating material.

5. The implantable device of claim 2, wherein the second lead further comprises a protective element at least partially surrounding the insulating material.

6. The implantable device of claim 5, wherein the protective element comprises a conductive material.

7. The implantable device of claim 6, wherein the reference electrode and the electrode conductor are electrically coupled via the protective element.

8. The implantable device of claim 5, wherein the reference electrode forms a terminus for the protective element.

9. The implantable device of claim 8, wherein the terminus comprises one of a metallic ring and a clamp.

10. The implantable device of claim 2, further comprising an additional insulating material partially surrounding the reference electrode such that a portion of the reference electrode is exposed by the additional insulating material.

11. The implantable device of claim 10, wherein a boundary between the exposed portion of the reference electrode and the insulating material comprises a smooth boundary.

12. The implantable device of claim 10, wherein the second lead further comprises a protective element that at least partially surrounds the lead conductor and the electrode conductor and is at least partially surrounded by each of the reference electrode and the insulating material.

13. The implantable device of claim 10, wherein the reference electrode comprises a mesh braid.

14. The implantable device of claim 1, wherein the second lead further comprises a portion of a connector for connecting to the separate module.

15. The implantable device of claim 14, wherein the reference electrode is located at an exterior surface of the portion of the connector.

16. The implantable device of claim 1, wherein the implantable device is configured to be implanted in a body.

17. The implantable device of claim 1, wherein the reference electrode comprises a bio-compatible conductive material.

18. The implantable device of claim 1, wherein the reference electrode comprises a noble metal.

19. The implantable device of claim 1, wherein the separate module comprises one or both of a power source and a microphone.

20. A lead for an implantable device, comprising:
a reference electrode configured to collect a stimulation current from at least one stimulation site;
a lead conductor configured to allow connection of a module to a stimulator;
an electrode conductor configured to conduct the stimulation current between the reference electrode and the stimulator; and
an insulating material surrounding at least the lead conductor and the electrode conductor.

21. The lead of claim 20, wherein the reference electrode is located at an exterior surface of the insulating material.

22. The lead of claim 21, wherein the reference electrode is patterned over at least a portion of the exterior surface of the insulating material.

23. The lead of claim 21, wherein the reference electrode is at least partially molded into the insulating material.

24. The lead of claim 21, further comprising a protective element at least partially surrounding the insulating material.

25. The lead of claim 20, further comprising an additional insulating material partially surrounding the reference electrode such that a portion of the reference electrode is exposed by the additional insulating material.

26. The lead of claim 20, further comprising a portion of a connector for connecting to the module.

27. The lead of claim 26, wherein the reference electrode is located at an exterior surface of the portion of the connector.

28. The lead of claim 20, wherein the module comprises one or both of a power source and a microphone.

29. A cochlear implant, comprising:
a stimulator configured to generate a stimulation current comprising a plurality of stimuli;
a first lead configured to conduct the stimulation current from the stimulator to at least one electrode;
a second lead configured to allow connection of an implantable module to the stimulator, wherein the second lead comprises (i) a lead conductor, (ii) a reference electrode configured to collect the stimulation current from the at least one electrode, (iii) an electrode conductor configured to conduct the stimulation current between the reference electrode and the stimulator, and (iv) a bio-compatible insulating material surrounding at least the lead conductor and the electrode conductor.

30. The cochlear implant of claim 29, further comprising the implantable module.

31. The cochlear implant of claim 29, wherein the implantable module comprises one of a power source and a microphone.
